(19) United States
(12) Patent Application Publication
(10) Pub. No.: US 2025/0152126 A1
(43) Pub. Date: May 15, 2025

(54) SYSTEMS AND METHODS FOR AUTOMATIC DETECTION AND LOCALIZATION OF FOREIGN BODY OBJECTS

(71) Applicant: THE JOHNS HOPKINS UNIVERSITY, Baltimore, MD (US)

(72) Inventors: Amir MANBACHI, Baltimore, MD (US); Haley Gilbert ABRAMSON, Baltimore, MD (US); Judy HUANG, Baltimore, MD (US); Henry BREM, Ellicott City, MD (US); Nicholas THEODORE, Ruxton, MD (US); George L. COLES, Baltimore, MD (US)

(21) Appl. No.: 18/836,900

(22) PCT Filed: Feb. 17, 2023
(86) PCT No.: PCT/US2023/013362

§ 371 Date: Aug. 8, 2024

Related U.S. Application Data
(60) Provisional application No. 63/311,926, filed on Feb. 18, 2022.

(51) Int. Cl. A61B 8/08; A61B 8/00; G06N 3/0464; G06N 3/082; G16H 30/40

(52) U.S. Cl. CPC A61B 8/0841; A61B 8/461; A61B 8/468; A61B 8/469; G06N 3/0464; G06N 3/082; G16H 30/40

(57) ABSTRACT

Systems and methods for detection and localization of a foreign body object in a region are provided. In embodiments, a system can a processor and one or more computer-readable storage media storing instructions which, when executed on the processor, cause the processor to perform a method for forming a trained model using a plurality of training images and a plurality of training boundary data sets. The systems and methods further apply the trained model to process an image and generate a boundary data set. In embodiments, the image can be an ultrasound image, and the boundary data set can correspond to a bounding box enclosing the foreign body object.

### CROSS REFERENCE TO RELATED APPLICATIONS

This application is the national stage entry of International Patent Application No. PCT/US2023/013362, filed on Feb. 17, 2023, and published as WO 2023/158834 A1 on Aug. 24, 2023, which claims the benefit of U.S. Provisional Patent Application No. 63/311,926, filed on Feb. 18, 2022, which are hereby incorporated by reference in their entireties.

### GOVERNMENT FUNDING

This invention was made with Government support under N66001-20-2-4075, awarded by Department of the Navy. The Government has certain rights in the invention.

### TECHNICAL FIELD

This disclosure relates to systems and methods for the automatic detection and localization of foreign body objects during and after surgery.

### BACKGROUND

Neurosurgical operations are long and intensive medical procedures during which the surgeon must constantly have an unobscured view of the brain to be able to properly operate. Currently, cotton balls are the most versatile and effective option to clear the view during surgery as they absorb fluids, are soft enough to safely manipulate the brain, and function as a spacer to keep anatomies of the brain open and visible during the operation. However, cotton may be retained post-surgery, which can lead to dangerous complications such as textilomas.

In addition to cotton balls, other foreign body objects, e.g. metal implants, stainless steel, latex glove fragments, and Eppendorf tubes, among other things, bring about very similar challenges to neurosurgery and other fields in medicine, and can result in risk to a patient's health and invasive reoperation.

### SUMMARY

Disclosed herein are methods, systems, and non-transitory computer readable media storing program instructions for localizing foreign body objects using ultrasound images.

Using ultrasound imaging, the different acoustic properties of cotton and brain tissue result in two discernible materials. Consistent with this disclosure, we created a fully automated foreign body object tracking algorithm that integrates into the clinical workflow to detect and localize retained cotton balls in the brain. This deep learning algorithm uses a custom convolutional neural network and achieves 99% accuracy, sensitivity, and specificity, and surpasses other comparable algorithms. Furthermore, the trained algorithm was implemented into web and smartphone applications with the ability to detect one cotton ball in an uploaded ultrasound image in under half of a second. Embodiments consistent with this disclosure also highlights the first use of a foreign body object detection algorithm using real in-human datasets, showing its ability to prevent accidental foreign body retention in a translational setting.

In one aspect, embodiments consistent with the present disclosure include a method of forming a trained model in which one or more processing devices perform operations including receiving a plurality of training images, each training image in the plurality of training images associated with a respective training boundary data set of a plurality of training boundary data sets, each training image in the plurality of training images further represented as a respective training image data set of a plurality of training image data sets. In embodiments, each of the plurality of training images is a respective ultrasound image of a plurality of ultrasound images, the respective ultrasound image further associated with a respective one of a plurality of regions. Further the operations can include processing the plurality of training image data sets using a convolutional neural network to generate a plurality of output boundary data sets and to select a plurality of training weights, where the convolutional neural network includes (i) a plurality of pre-trained layers with a plurality of pre-trained weights, and (ii) a plurality of training and appended layers with the plurality of training weights, and where, when using the convolutional neural network to generate the plurality of output boundary data sets, the plurality of pre-trained weights are fixed and the plurality of training weights are selected to minimize a loss function between the plurality of output boundary data sets and the plurality of respective training boundary data sets. In embodiments, the operations can include fixing the plurality of training weights to form the trained model when the loss function is minimized, where fixing the plurality of training weights further selects a plurality of fixed training weights.

In a further aspect, a method of generating a boundary data set from an input image in which one or more processing devices perform operations can include forming a trained model, receiving the input image represented as an input image data set, and processing the input image data set using the convolutional neural network to generate the boundary data set. In an aspect, the convolutional neural network can include (i) the plurality of pre-trained layers with the plurality of pre-trained weights, and (ii) the plurality of training and appended layers with the plurality of fixed training weights.

In another aspect, a system for forming a trained model consistent with the present disclosure can include a non-transitory computer readable storage medium associated with a computing device, the non-transitory computer readable storage medium storing program instructions executable by the computing device to cause the computing device to perform operations including receiving a plurality of training images, each training image in the plurality of training images associated with a respective training boundary data set of a plurality of training boundary data sets, each training image in the plurality of training images further represented as a respective training image data set of a plurality of training image data sets. In an aspect, each of the plurality of training images is a respective ultrasound image of a plurality of ultrasound images, the respective ultrasound image further associated with a respective one of a plurality of regions. Further, in an aspect consistent with the disclosure, the operations can include processing the plurality of training image data sets using a convolutional neural network to generate a plurality of output boundary data sets and to select a plurality of training weights, where the convolutional neural network includes (i) a plurality of pre-trained layers with a plurality of pre-trained weights, and (ii) a plurality of training and appended layers with the plurality of training weights, and when using the convolutional neural network to generate the plurality of output boundary data sets, the plurality of pre-trained weights are fixed and the plurality of training weights are selected to minimize a loss function between the plurality of output boundary data sets and the plurality of respective training boundary data sets. In an aspect, the operations can include fixing the plurality of training weights to form the trained model when the loss function is minimized, where fixing the plurality of training weights further selects a plurality of fixed training weights.

In another aspect, a system consistent with the present disclosure include at least one processor, and at least one non-transitory computer readable media associated with the at least one processor storing program instructions that when executed by the at least one processor cause the at least one processor to perform operations for generating a boundary data set from an input image, where the operations include: receiving the input image represented as an input image data set; and processing the input image data set using a convolutional neural network to generate the boundary data set. In an aspect, the convolutional neural network can include (i) a plurality of pre-trained layers with a plurality of pre-trained weights, and (ii) a plurality of training and appended layers with a plurality of fixed training weights, where the plurality of fixed training weights are selected according to training operations performed by one or more processors associated with one or more non-transitory computer readable media, the one or more non-transitory computer readable media storing training program instructions that when executed by the one or more processors cause the one or more processors to perform the training operations. In an aspect, the training operations can include receiving a plurality of training images, each training image in the plurality of training images associated with a respective training boundary data set of a plurality of training boundary data sets, each training image in the plurality of training images further represented as a respective training image data set of a plurality of training image data sets, where each of the plurality of training images is a respective ultrasound image of a plurality of ultrasound images, the respective ultrasound image further associated with a respective one of a plurality of regions. The training operations can further include processing the plurality of training image data sets using a training convolutional neural network to generate a plurality of output boundary data sets and to select a plurality of training weights, where the training convolutional neural network include (i) the plurality of pre-trained layers with the plurality of pre-trained weights, and (ii) the plurality of training and appended layers with the plurality of training weights. In an aspect, when using the training convolutional neural network to generate the plurality of output boundary data sets, the plurality of pre-trained weights are fixed and the plurality of training weights are selected to minimize a loss function between the plurality of output boundary data sets and the plurality of respective training boundary data sets. Further, the training operations can include fixing the plurality of training weights to form the trained model when the loss function is minimized, where fixing the plurality of training weights further selects the plurality of fixed training weights.

In further aspects, the loss function can be a mean squared error function using a plurality of error values, each error value of the plurality of error values being equal to a respective numerical difference between at least one of the plurality of output boundary data sets and a respective one of the plurality of training boundary data sets. Additionally, at least one of the plurality of ultrasound images associated with a respective one of the plurality of regions is further associated with a respective foreign body object in the respective one of the plurality of regions. Further, the at least one of the plurality of ultrasound images associated with the respective one of the plurality of regions can be further associated with a respective one of the plurality of training boundary data sets, such that the respective one of the plurality of training boundary data sets is a set of number values associated with a bounding box enclosing the respective foreign body object. In embodiments, the bounding box enclosing the respective foreign body object can be a ground truth bounding box.

In other aspects, at least one of the plurality of ultrasound images is associated with a respective one of the plurality of regions such that the respective one of the plurality of regions contains no foreign body object. In further embodiments, at least one of the plurality of ultrasound images associated with the respective one of the plurality of regions containing no foreign body object is further associated with a respective one of the plurality of training boundary data sets, such that the respective one of the plurality of training boundary data sets is a set of number values associated with a null bounding box. In another aspect, the set of number values associated with the null bounding box can include: an x-coordinate value equal to zero of the null bounding box; a y-coordinate value equal to zero of the null bounding box; a width value equal to zero of the null bounding box; and a height value equal to zero of the null bounding box.

In other aspects, the set of number values associated with the bounding box enclosing the respective foreign body object can include: an x-coordinate value of an upper left corner of the bounding box; a y-coordinate value of the upper left corner of the bounding box; a width value of the bounding box; and a height value of the bounding box.

In further aspects, the convolutional neural network can include a VGG16 model, and the input image can be an ultrasound image of a region associated with a potential foreign body object in the region.

In other aspects, each respective foreign body object can include at least one of: a cotton ball, a stainless steel rod, a latex glove fragment, an Eppendorf tube, a suturing needle, and a surgical tool.

Further still, in an aspect, the plurality of appended layers can include at least one of: a dropout layer and a dense layer.

In further embodiments, operations consistent with this disclosure can include generating a representation for display on a display device, the representation for display including an overlay of a representation of the output bounding box on a representation of the input image.

In further embodiments, a smartphone can include the at least one processor, the at least one non-transitory computer readable media, and the display device. In other embodiments, the smartphone can be configured to capture the input image.

In another embodiment, a networked computer device can include the at least one processor and the at least one non-transitory computer readable media, and a remote computing device can include the display device and can be configured to transmit the input image to the networked computing device. In further embodiments, the remote computing device is further configured to capture the input image.

Additional features and embodiments of the invention will be set forth in part in the description which follows, and in part will be obvious from the description, or may be learned by practice of the invention. It is to be understood that both the foregoing general description and the following detailed description are exemplary and explanatory only and are not restrictive of the claimed subject matter.

### BRIEF DESCRIPTION OF THE DRAWINGS

The accompanying drawings, which are incorporated in and constitute a part of this specification, illustrate exemplary embodiments and together with the description, serve to explain the principles of the disclosure. In the figures:

FIG. 1 depicts an exemplary system consistent with this disclosure;

FIG. 2 depicts an exemplary foreign body object detection in neurosurgery consistent with this disclosure;

FIG. 3 depicts a setup for both animal studies and in-human studies;

FIG. 4 depicts the architecture of a convolutional neural network consistent with this disclosure;

FIG. 5 depicts exemplary methods consistent with embodiments of this disclosure;

FIG. 6 depicts the use of intersection over union (IoU) consistent with this disclosure;

FIG. 7 depicts the use of other algorithms for foreign body object detection (A-D) compared to the use of embodiments consistent with this disclosure (E-G);

FIG. 8 depicts an accuracy assessment based on cotton ball size using embodiments consistent with this disclosure;

FIG. 9 depicts exemplary predictions for embodiments consistent with this disclosure;

FIG. 10 depicts an acoustic comparison of cotton balls soaked with saline (A) and cotton balls soaked with Doppler fluid (B);

FIG. 11 depicts further exemplary foreign body object detection consistent with this disclosure;

FIG. 12 depicts additional exemplary foreign body object detection consistent with this disclosure; and

FIGS. 13-14 depict further embodiments consistent with this disclosure.

### DESCRIPTION OF THE EMBODIMENTS

Reference will now be made in detail to the disclosed embodiments of the disclosure, examples of which are illustrated in the accompanying drawings. Wherever possible, the same reference numbers will be used throughout the drawings to refer to the same or like parts.

While cotton is used to absorb blood during neurosurgical procedures, and may become visually indistinguishable from the surrounding tissue, it has distinct acoustic properties from the brain parenchyma that can be picked up safely and effectively with ultrasound imaging.

Disclosed herein are systems and methods that use ultrasound technology in order to minimize foreign object retention during and after surgical procedures and reduce undesired post-operative risks. In one embodiment, systems and methods consistent with this disclosure use automated deep learning technology for the localization of cotton balls during and/or after neurosurgery, and take advantage of the unique acoustic properties of cotton and the ability of deep neural networks to learn specified image features. In other embodiments, consistent with this disclosure, systems and methods use automated deep learning technology for the localization of foreign body objects (e.g., metal implants, stainless steel, latex glove fragments, and Eppendorf tubes, among other things) during and or after surgery.

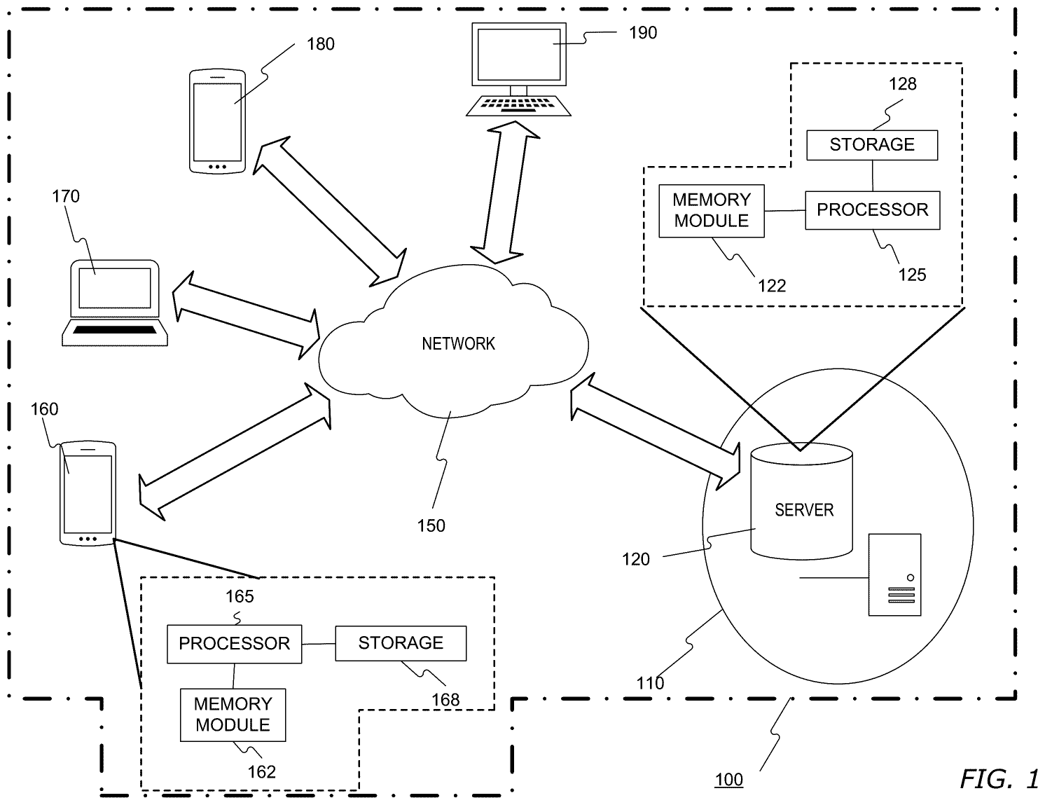

FIG. 1 depicts system 100, which includes computer network components consistent with embodiments. System 100 can include server 110, which can be accessed over network 150 and can operate a convolutional neural network model consistent with this disclosure. Remote clients 160, 170, 180, and 190 can access server 110 over network 150. Also shown are processor 165, memory module 162, and storage 168 associated with remote client 160, and processor 125, memory module 122 and storage 128 associated with server 120. Consistent with certain embodiments, remote client 160 can also operate a convolutional neural network consistent with this disclosure (as could remote clients 170, 180, and 190). Other comparable components in the remaining remote clients are not shown.

### Introduction

Leaving behind surgical items in the body is considered a “never event” [1], yet it markedly burdens both patients and hospitals with millions of dollars spent every year on medical procedures and legal fees, costing $60,000 to $5 million per case [2-4]. Nearly 14 million neurosurgical procedures occur annually worldwide [5], and in each craniotomy surgeons may use hundreds of sponges or cotton balls to clear their field of view. Thus, it is unsurprising that surgical sponges are the most commonly retained items [6]. Unfortunately, retained foreign body objects may lead to life-threatening immunologic responses, require reoperation, or cause intracranial textilomas and gossypibomas, which mimic tumors immunologically and radiologically [7-10]. Locating cotton balls on or around the brain becomes increasingly challenging as they absorb blood, rendering them visually indistinguishable from the surrounding tissue. Unlike larger gauze pads, which are often counted using radiofrequency tagged strips [11], cotton balls are small (closer to 10 mm in diameter), must be counted manually by nurses in the operating room as they are placed in and extracted from the open wound, and may leave behind a small torn strip of cotton. There is therefore an unmet need for an intraoperative, automatic foreign body object detection solution that can be streamlined into the neurosurgical workflow. Due to their prevalence in surgical procedures and the difficulties associated with tracking their use, cotton balls serve as an excellent model of retained foreign bodies inside the cranial cavity.

Although seeing the contrast between blood-soaked cotton balls and brain tissue poses a challenge, they can be distinguished by listening to them. Prior work has demonstrated that ultrasound is able to capture the different acoustic characteristics between these materials and interpret them via filtering and logarithmic compression to display distinctly on an ultrasound image [12]. More specifically, ultrasound captures the difference in the acoustic impedance between brain parenchyma and cotton as a result of their distinct densities and the speed at which sound travels through them (acoustic impedance is the product of material density and speed of sound). Ultrasound is non-invasive, nonradiating, clinically available, inexpensive, portable, and able to display images in real time. Therefore, ultrasound is an optimal modality for visualizing and localizing retained cotton during neurosurgery.

Deep learning (DL) has shown promise in object localization within an image [13]; therefore, a DL algorithm using ultrasound images holds exceptional potential as a solution to fill this clinical need. However, medical images have notably high resolution, complexity, and variability as a result of alternative patient positions and respiration artifacts. In general, ultrasound is widely considered as a relatively difficult imaging modality to read, as specialized sonographers must undergo years of training for certification to operate clinical machines, and the same goes for trained radiologists to read these images. Hence, DL with medical ultrasound images (e.g., diagnosis, object detection, etc.) can be particularly complicated and computationally expensive. A previous DL approach to cotton ball detection used a model called YOLOv4 [14] and reported an accuracy of 60% [15].

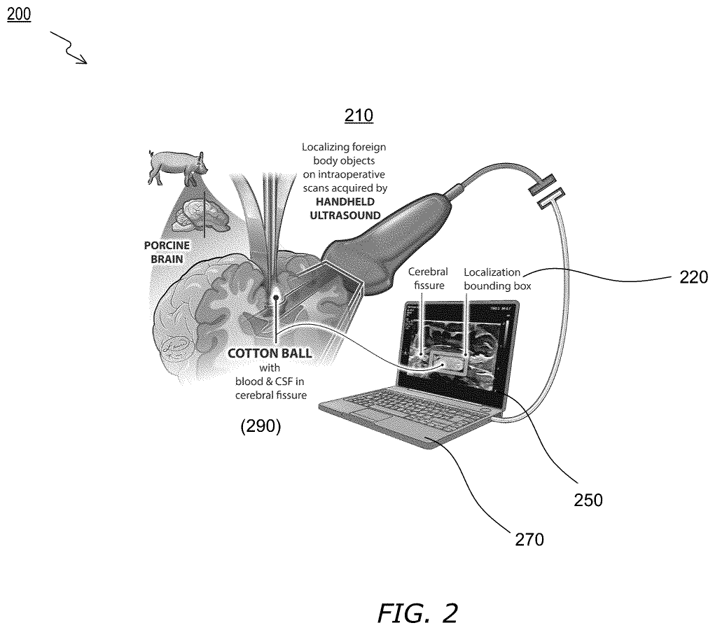

Here we present a highly accurate (99%), rapid (<0.5 s) ultrasound-based technology for both detection and localization of retained cotton in brain tissue. An exemplary system 200 is depicted in FIG. 2, which can include handheld ultrasound 210 and computer 270 (which can be a standalone or networked computer). Also shown is exemplary foreign body object 290, which is a cotton ball. Consistent with this disclosure, an ultrasound image can be generated using the handheld ultrasound 210, and computer 270 can be configured to acquire that ultrasound image directly or through other means (such as uploading the ultrasound image over a network, acquiring a screenshot, or taking a photo of a screen displaying the ultrasound image). Computer 270 can either process the acquired input ultrasound image using a convolutional neural network, consistent with those disclosed herein, or it can communicate with a remote computer (not shown) that is configured to process the input ultrasound image as described herein on order to generate a bounding box 220, which can be configured to overlay the input image in order to produce an output representation 250. Embodiments consistent with this disclosure demonstrate the ability of medical ultrasound to locate foreign bodies, specifically cotton balls 290, retained in the brain post-surgery.

We demonstrate the necessity of its inclusion in clinic via human studies, as a cotton ball 290 not initially visible to a neurosurgeon in the surgical site was clearly observed in ultrasound images and subsequently removed from the cavity. This algorithm was able to identify that cotton ball. Ultrasound images acquired using a clinical ultrasound machine may be loaded into a web application hosted on a local server or captured with a smartphone camera using a custom app, which both reach a trained deep neural network that performs nonlinear regression and outputs the same image with one bounding box enclosing a cotton ball (if one exists). By localizing retained surgical objects within an ultrasound image, this method can distinguish between small fragments of cotton and folds in the brain parenchyma. It also could bring ease during long and intensive surgeries by alerting a clinician who may not be trained in sonography to a particular region of interest in an image, thus acting as an assistive device. First ever in-human studies show that this algorithm is already clinically relevant and ready to be incorporated seamlessly into neurosurgeries, with broad implications in medicine. Embodiments consistent with this disclosure pave the path for improved patient outcomes, minimal surgical errors, and reduction of the need for revisionary procedures and associated healthcare costs.

### Materials and Methods, Data Acquisition, Animal Setup

The algorithm used with embodiments consistent with this disclosure was developed and tested using ex vivo porcine brain images. Porcine brains (Wagner Meats, Maryland, USA) were obtained and imaged with implanted cotton balls within 24 h of euthanasia. Prompt post-mortem imaging was necessary to avoid transformations in the acoustic properties of the brain tissue [16], which would change how the ultrasound machine interprets the image. These brain samples (N ¼ 10) were placed in a rubber-lined acrylic container filled with 1×pH 7.4 phosphate-buffered saline (PBS, ThermoFisher) to minimize artifacts in the recorded images. For imaging, an eL18-4 probe was used with a Philips EPIQ 7 (Philips, Amsterdam, Netherlands) clinical ultrasound machine.

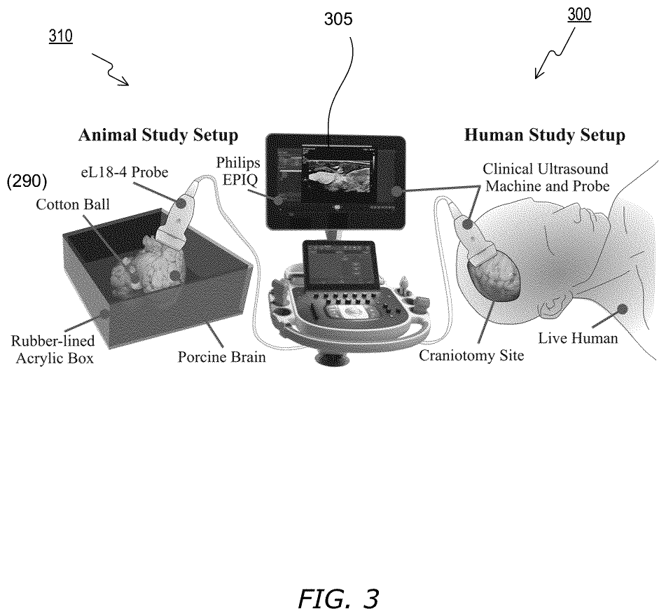

FIG. 3 depicts an experimental setup 310 for conducting animal studies and an experimental setup 300 for conducting in-human studies. Animal images (ultrasound images 305) were acquired by setting an rubber-lined acrylic box, placing cotton balls 290 within the brain, and immersing in saline. An eL 18-4 probe of a Philips EPIQ 7 ultrasound machine was used to capture these ultrasound images 305 (with the experimental setup for animal studies depicted on the left-hand side of FIG. 3). The human studies were performed during neurosurgical procedures using an Aloka Alpha 7 machine and UST-9120 probe (with the experimental setup for in-human studies depicted on the right-hand side of FIG. 3). The cranial cavity was scanned for retained cotton balls 290.

Different sizes and locations of the cotton balls 290 were imaged to mimic a neurosurgical procedure, including a control group with no cotton balls 290. Cotton balls 290 were trimmed to diameters of 1, 2, 3, 5, 10, 15, and 20 mm. Approximately the same number of still images 305 were captured for each size of cotton ball 290, with more control images 305 to stress the importance of recognizing true negatives (i.e., understanding when there is not a cotton ball 290 in the image 305). One saline-soaked cotton ball 290 was implanted in the porcine brain per true positive image. To improve the variability among the images 305, the cotton ball 290 was implanted at depths between 0 mm (placed directly underneath the transducer, above the brain) to approximately 40 mm (placed at the bottom of the container, beneath the brain). During imaging, the probe was moved and rotated around the outer surface of the brain to provide additional variability in the location of the cotton ball 290 in the ultrasound image 305.

Additionally, experiments ensured that the acoustic properties of cotton in an ex vivo setting were representative of an in vivo setting, i.e., when soaked in blood during neurosurgery. Ultrasound imaging compared a 20 mm diameter cotton ball 290 soaked in PBS with one soaked in Doppler fluid (CIRS, Norfolk, VA, USA, Model 769DF). Doppler fluid is designed to mimic the acoustic properties of blood. These images were compared visually by eye and by average pixel intensity value of the cotton ball 290, which would help ensure the DL algorithm could recognize cotton retained in an in vivo setting. The acoustic properties of cotton, PBS, and Doppler fluid were also assessed to confirm that the images 305 should look similar based on the equation for acoustic impedance, which is used by the ultrasound machine to translate sound waves into image pixels.

Finally, other materials were tested using the technology developed here as well. A latex glove fragment (5 mm diameter), a stainless steel rod (5 mm diameter and 18 mm length), and an Eppendorf tube (7 mm in diameter and 30 mm in length) were placed on or around a porcine brain, imaged using ultrasound, and tested using the same methods as the cotton balls 290.

### Materials and Methods, Data Acquisition, In Vivo Human Studies

Ultrasound images 305 of live human brains (N ¼ 2) were captured prior to closing the cranial cavity following (1) an aneurysm surgery and (2) a glioblastoma tumor resection. These images were acquired as part of a standard protocol by the neurosurgeon. Images 305 were de-identified prior to being provided for evaluation of cotton ball 290 presence, and this evaluation was conducted post-operatively (i.e., not as a part of the surgery).

The ultrasound machine available to the operating rooms was the Aloka Prosound Alpha 7 with a UST-9120 probe (Hitachi Aloka Medical America, Inc., Wallingford, CT). For the purposes of embodiments consistent with this disclosure, a 10 mm diameter cotton ball 290 was momentarily placed in the location of suturing or tumor removal, and saline was used to eliminate potential air bubbles prior to capturing the ultrasound images 305, which proceeded as follows. First, the neurosurgeon tasked with acquiring the ultrasound images 305 identified the region of interest, i.e., the surgical site where a foreign body was known to have been placed. In a general case without a known cotton ball 290 placement, this region of interest would be the open cranial cavity. The ultrasound probe was placed at the start of this window, with the depth adjusted to avoid image artifacts due to skull bone as depicted in FIG. 3. During these surgeries, cine (moving) image scans were captured and later saved slice-wise as still images, although still images may be acquired as these are the input images to the custom algorithm associated with embodiments consistent with this disclosure. The probe was tilted in increments of 15, taking care again to avoid bone reflections. To ensure that the entire region of interest is checked for cotton ball 290 presence, the neurosurgeon translated the probe across the contour of the anatomy. The neurosurgeon slid the probe in 1 cm increments in both the anterior-posterior and lateral directions until the entire exposed surface of the brain was covered. Once the cotton ball 290 intentionally placed in the cavity was removed, a follow-up ultrasound scan captured another set of true negative images 305. During the second patient's procedure, another cotton ball 290 was visible on the ultrasound images 305. Thus, that cotton ball was found and removed in order to acquire images absent of any foreign body objects. Later, the de-identified images annotated by clinicians were checked for presence of a cotton ball 290 using the developed web application.

### Materials and Methods, Data Preprocessing

All images were annotated with ground truth bounding boxes surrounding cotton within the brain by researchers who conducted the studies shown here. The ground truth porcine brain images 305 served as data for the DL model, split randomly but evenly by cotton ball 290 diameter into 70% training set, 15% validation set, and 15% test set. Images 305 were processed using anisotropic diffusion, which emphasizes edges while blurring regions of consistent pixel intensity, scaled from (768, 1024, 3) to (192, 256, 3) to decrease the computational power required to process each image, and normalized to pixel intensity values between 0 and 1. Each pixel in an image has an associated red, green, and blue color value, thus lending to the 3-dimensionality of the image. Intraoperative neurosurgical images in humans captured with a lower resolution probe additionally underwent contrast-limited adaptive histogram equalization (CLAHE) with a clip limit of 2.0 and a tile grid of side length 4 to increase image contrast.

### Materials and Methods, Algorithmic Design

To ensure DL was in fact the optimal method for localizing cotton balls 290 within ultrasound images 305, multiple less computationally expensive methods were implemented for comparison. These included thresholding and template matching. Because cotton appears brighter than most brain tissue in ultrasound images, an initial threshold at half the maximum of all grayscale pixel values was attempted [17]. Additionally, Otsu thresholding was implemented as a method for identifying a natural threshold in the image [18]. Finally, the average pixel values within ground truth bounding boxes of the training set images were calculated, and the images in the test set were thresholded at the 95th percentile of these averages. To implement template matching, four examples of different cotton balls 290 were cropped from training set images to serve as “templates.” These template images were moved across each image of the test set at various scales from 25% to 200% the size of the template, and the location with the highest correlation value (most similar pixels) was taken to be the location of the cotton ball in the test set image [19]. As an additional method for comparison, CSPDarknet53 [20], the DL backbone of YOLOv4 used in Mahapatra et al. [15], was implemented.

Ultimately, a fully automated DL algorithm for object localization was developed and packaged in a web application. DL is implemented in the form of neural networks, which are series of differentiable functions called “layers” that transform an input into a desired output. Convolutional neural networks (CNNs) are tailored towards image analysis. A CNN known as VGG16 [21] has shown success at reducing large medical image files to a few meaningful numbers. Here, a custom version of this model was used to predict four numbers from each ultrasound image 405 representing: (1) the x value of the top left corner of the annotated bounding box, (2) the y value of the top left corner of the bounding box, (3) the width of the bounding box, and (4) the height of the bounding box.

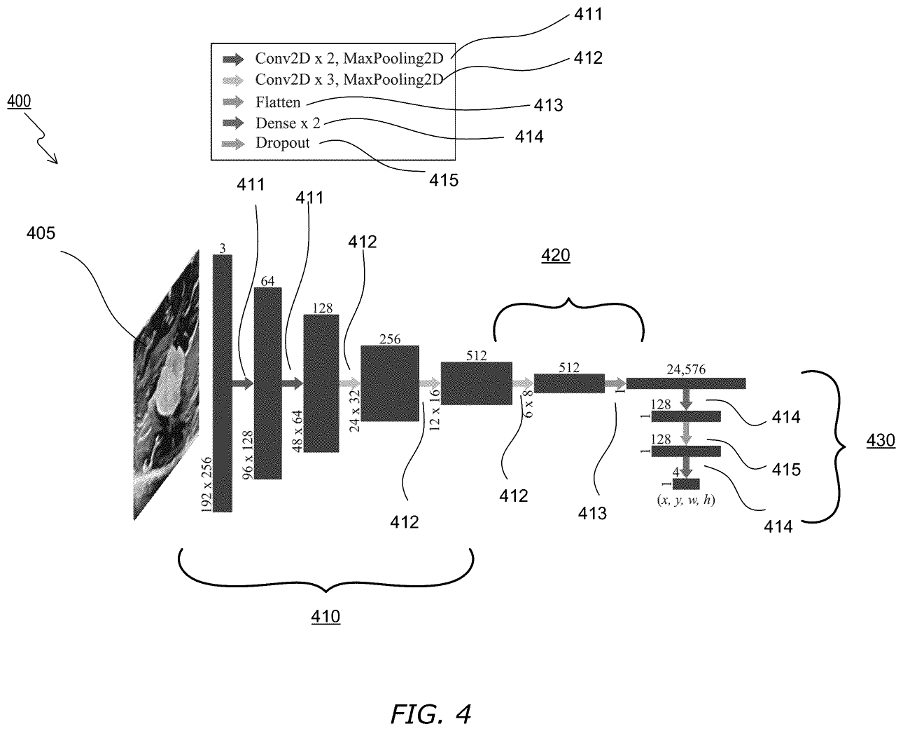

The VGG16 model was customized by fine-tuning it and appending additional layers 430. When fine-tuning, pre-trained weights are used throughout most of the model (layers 410) except for the final few layers (layers 420, referred to as training layers 420). These weights tell the network what to look for in an image. In a typical neural network, the initial layers tend to look more broadly at curves and lines, while the latter layers are trained to recognize high-level features specific to the task at hand, such as textures and shapes. By learning new weights for the last few layers, the network is able to be applied to new tasks; this process is known as fine-tuning [22]. Thus, the network designed here implemented VGG16 using ImageNet weights (which are included in the Keras DL package [23]) for all layers except the last four layers 420 (training layers 420), which remained “unfrozen,” or trainable. Additionally, five layers 430 (appended layers 430) were appended to the VGG16 network: four dense layers split by a dropout layer. This configuration of a custom convolutional neural network 400 is depicted in FIG. 4.

FIG. 4 depicts the architecture of custom convolutional neural network 400 consistent with this disclosure. A convolutional neural network known as VGG16 was used as a backbone with an additional dense network split with a layer of dropout. Vertically and horizontally displayed number values indicate the 3-dimensional sizes of each layer (represented in FIG. 4 as solid rectangles). The input is a 192×256×3 array (where the depth of 3 represents red, green and blue values assigned to each pixel) that is transformed to an output that is 1×4. These 4 values represent the x and y coordinates of the upper left corner of the predicted bounding box as well as the width and height of the box. Each arrow represents a function applied to the preceding layer as indicated in the legend. Conv2D×2, MaxPolling 2D function is indicated with reference number 411; Conv2D×3, MaxPolling 2D function is indicated with reference number 412; Flatten function is indicated with reference number 413; Dense×2 function is indicated with reference number 414 and Dropout function is indicated with reference number 415. Convolutional layers were implemented with a 3×3 kernel size; max pooling layers used a 2×2 stride size; and dropout was 50%.

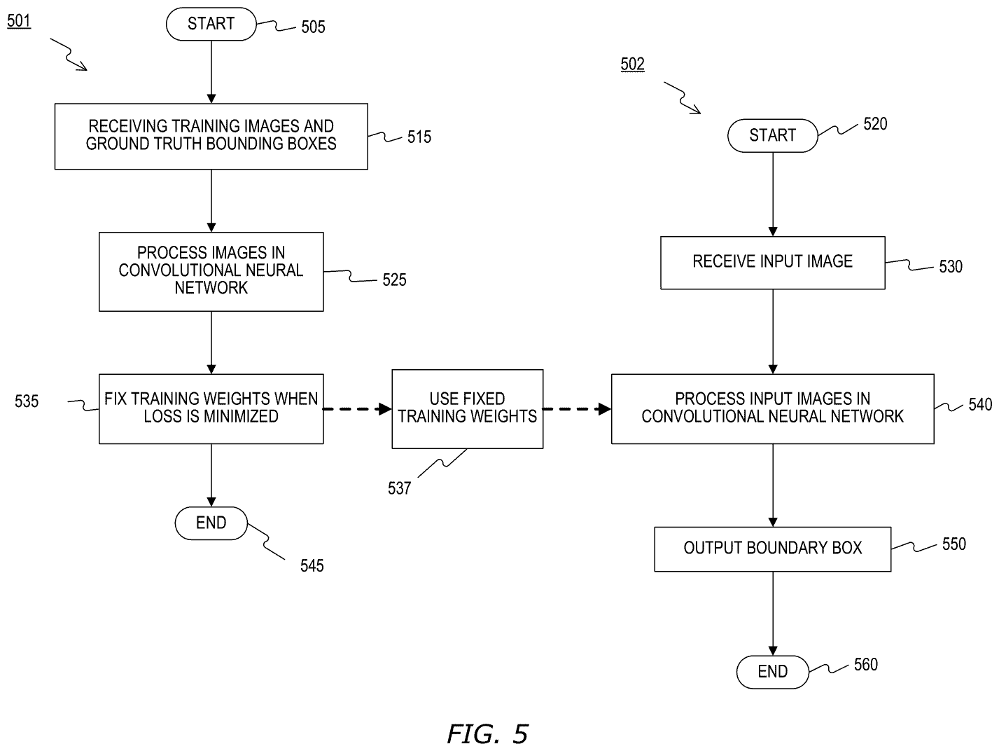

FIG. 5 depicts general methods consistent with the disclosure, involving a training procedure 501 and an image processing procedure 502. As described in further detail below, training procedure 501 begins (step 505) by receiving a plurality of training images 405 and ground truth bounding boxes (step 515). As used herein, the data associated with a ground truth bounding box associated with one training image 405 will be referred to as a training boundary data set. The plurality of training images 405 and plurality of training boundary data set values are processed in the customized convolutional neural network 400 as described further above and below. One of ordinary skill in the art, for example, would appreciate that a subset of weights used in customized convolutional neural network 400 can be varied so as to minimize a loss function as the customized convolutional neural network processes the plurality of training images 405 (step 525). This subset of weights, referred to herein as training weights, are fixed when the loss is minimized for the plurality of training images 405 (step 535). At this point, the customized convolutional neural network 400 is considered a trained model (step 545).

Further still, as described further below, image processing procedure 502 begins (step 520) by receiving an input image (step 530). The input image is processed by the customized convolutional neural network as a trained model (step 540). Specifically, the plurality of fixed training weights determined according to training procedure 501 are conveyed to image processing procedure 502. The output of image processing procedure 502 will be a boundary data set, that will be associated with a predicted bounding box and any foreign body object that may be associated with the region in the ultrasound image (step 550). This concludes the image processing procedure 502 (step 560). As depicted in FIG. 5, the fixed training weights from procedure 501 are used in procedure 502 (step 537).

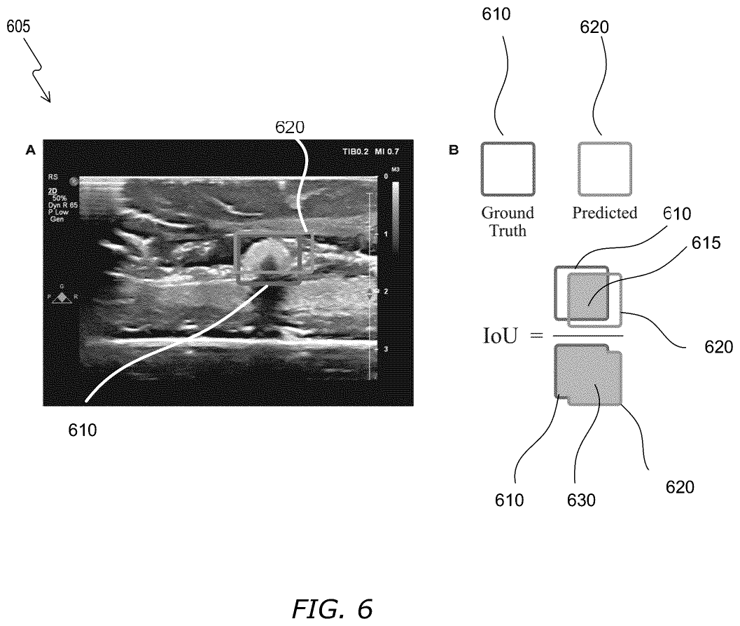

Returning to the architecture depicted in FIG. 4, all convolutional layers and the first three dense layers used ReLU activation [22], while the final dense layer used sigmoid activation [22]. The dropout layer was implemented with 50% dropout. Unlike convolutional layers, which learn local patterns, dense layers are fully connected and therefore learn global patterns in the data. Dropout layers help to regularize the model, i.e., improve its ability to generalize across images beyond the training set [22]. A data generator shuffled the training set as it prepared batches of 64 images for each training step. The network passed each of the training set images through its layers 50 times, where each pass through the set is known as an epoch. Each of the 50 epochs had [n_{imgs}/64] steps. An Adam optimizer was implemented with a learning rate of 0.001. This parameter indicates the amount by which the weights in the model can change after each epoch, where a smaller learning rate also implies that the network learns more slowly. The loss function used to train and optimize the network was mean squared error. Results were evaluated using the intersection over union (IoU), which is the ratio of the overlap in predicted and ground truth bounding boxes to the combined total area of these boxes, and is shown in FIG. 6.

Specifically, FIG. 6A depicts ultrasound image 605 with ground truth bounding box 610 (as annotated) and predicted bounding box 620 from the customized convolutional network 400. To the right of the image 605, (i.e., FIG. 6B) IoU is shown as intersection (615) over union (630). Specifically, the extent of overlap is assessed by computing the area of overlap (615) divided by area of union (630), known as the IoU.

An accurate prediction was considered one with an IoU over 50% [25]. In addition to running the custom network on the randomly assigned training, validation, and test sets described above, stratified 5-fold cross validation (CV) was implemented to avoid overfitting. This method divided the entire set of images collected into five groups with randomly but evenly distributed cotton ball sizes. Each group took a turn as the test set, with the other four serving together as the training set. Mean IoU, accuracy, sensitivity, and specificity were calculated for each of the five models trained and averaged to get a final, cross-validated result. CV was performed on each of the compared neural networks. Gradio [26], a Python (RRID:SCR_008394) package, was used to develop an intuitive web-based interface that fits into the clinical workflow. The smartphone application was designed using Flutter, powered by Dart [27]. All training was performed using a NVIDIA RTX 3090 GPU using Keras and Tensorflow (RRID:SCR_016345).

### Results

In addition to highly accurate cotton ball 290 detection in ex vivo porcine brains, the trained algorithm was able to detect cotton balls 290 in in vivo human studies and other medical foreign objects placed in an ex vivo setting. This algorithm has demonstrated its importance in human surgery by locating a cotton ball that was then removed from a patient, not having been known to exist prior to imaging as it was visually indistinguishable from surrounding brain tissue.

The acquired dataset of ex vivo porcine brain ultrasound images 405 was large and diverse, both of which are necessary qualities for a successful deep learning model. In total, 7,121 images were collected from 10 porcine brains. Table 1 provides a more detailed breakdown.

TABLE 1
Number of images acquired of each cotton ball
for training, validation, and testing of the model.

| Cotton ball diameter (mm) | No. of images |
|---|---|
| 0 mm | 1,456 |
| 1 mm | 986 |
| 2 mm | 878 |
| 3 mm | 622 |
| 5 mm | 773 |
| 10 mm | 820 |
| 15 mm | 825 |
| 20 mm | 641 |
| Total | 7,121 |
| Training | 4,898 |
| Validation | 1,046 |
| Testing | 1,057 |

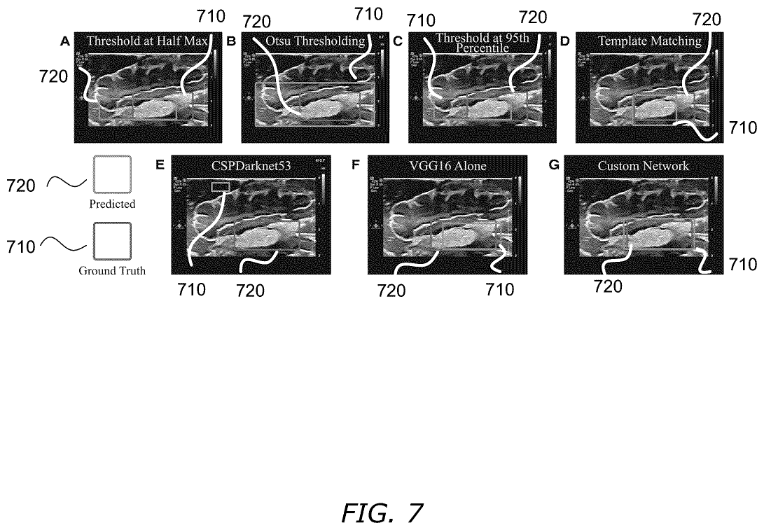

Thresholding and template matching methods that were implemented as control algorithms to verify the necessity for DL were performed both with and without images where a cotton ball 290 was present (i.e., true negatives were either included or excluded). These non-DL methods would likely always report a cotton ball 290 existing, so true negatives were excluded to ensure comparison to the best possible results of thresholding and template matching. However, results including and excluding true negatives for these non-DL methods are both shown for robustness. Specificity is unable to be calculated in cases where true negatives do not exist. Results of each algorithm are displayed in Table 2 and FIG. 7.

TABLE 2
Control algorithms included thresholding and template matching methods.
The threshold at the 95th percentile was calculated using the bounding boxes of ground truth
images in the training set. These control algorithms were either performed with or without true
negative (TN) images, i.e., images known not to contain a cotton ball. The YOLOv4 backbone,
CSPDarknet53, was implemented for comparison as well. Our custom network is a VGG16
backbone with an additional Dense network. Each neural network was additionally
implemented and evaluated using stratified 5-fold cross validation (CV). The final custom
algorithm achieved a mean IoU of 0.92, or 0.94 after CV.

| Algorithm | TN images? | Mean IoU | Sensitivity | Specificity | Accuracy |
|---|---|---|---|---|---|
| Threshold at half | Yes | 0.17 | 0.21 | 0.0 | 0.16 |
| maximum | | | | | |
| Threshold at half | No | 0.41 | 0.39 | — | 0.39 |
| maximum | | | | | |
| Otsu Thresholding | Yes | 0.11 | 0.06 | 0.0 | 0.04 |
| Otsu Thresholding | No | 0.21 | 0.09 | — | 0.10 |
| Threshold at 95th | Yes | 0.18 | 0.24 | 0.0 | 0.19 |
| percentile | | | | | |
| Threshold at 95th | No | 0.46 | 0.48 | — | 0.49 |
| percentile | | | | | |
| Template matching | Yes | 0.07 | 0.003 | 0.0 | 0.003 |
| Template matching | No | 0.19 | 0.008 | — | 0.008 |
| CSPDarknet53 | Yes | 0.50 | 0.56 | 0.0 | 0.46 |
| (YOLOv4 backbone) | | | | | |
| 5-fold CV of | Yes | 0.52 | 0.77 | 0.0 | 0.62 |
| CSPDarknet53 | | | | | |
| VGG16 alone | Yes | 0.89 | 0.99 | 0.98 | 0.99 |
| 5-fold CV of VGG16 | Yes | 0.52 | 0.40 | 0.60 | 0.45 |
| Custom network | Yes | 0.92 | 0.99 | 0.99 | 0.99 |
| S-fold CV of custom | Yes | 0.94 | 1.0 | 1.0 | 1.0 |
| network | | | | | |

Algorithm comparison is shown in FIGS. 7A-G. FIGS. 7A-D depict the output of non-deep learning algorithms implemented to predict the location of a cotton ball 290 in a neurosurgical ultrasound image. These included (A) Thresholding at half the maximum pixel intensity value; (B) Otsu thresholding; (C) Thresholding at the 95th percentile of the average pixel value within the ground truth bounding box of a training set; and (D) Matching the input image to a template image of a cotton ball. In each figure, ground truth bounding box 710 is depicted and predicted bounding box 720. FIG. 7E depicts the output of CSPDarknet53 consistent with this disclosure, which is the backbone of the YOLOv4 algorithm, and which is known for object detection and classification. FIG. 7F depicts the output of VGG16 consistent with this disclosure, which is often employed for object localization and was implemented as in [29]. FIG. 7G depicts the output of the custom network 400 was employed using a VGG16 backbone and additional dense network as described and consistent with this disclosure.

Given that an accurate result is defined here as one with an IoU greater than 50% [25], no control algorithm reached a mean IoU that could be considered accurate without the use of DL. The neural network backbone commonly used in YOLOv4 implementations, CSPDarknet53, surpassed this threshold by 2% using stratified 5-fold CV. The standard VGG16 network without our customization also resulted in a mean IoU of 0.52 using CV.

Ultimately, the tailored network using a VGG16 backbone and custom dense network described above (network 400) reached both sensitivity and specificity values of 99% on a hold-out test set.

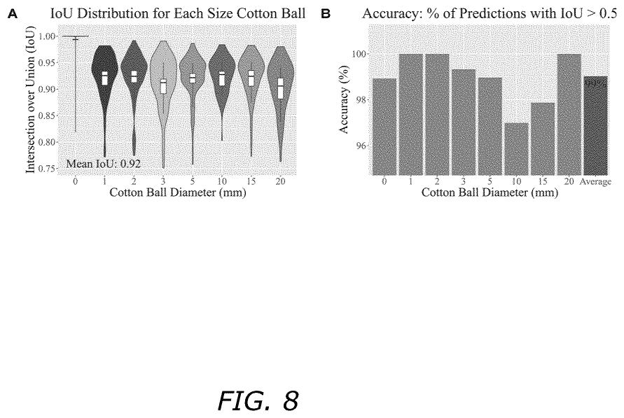

It also resulted in a median IoU of 94%+0:09 and mean IoU of 92% on this test set, as shown in FIG. 8A. Importantly, the algorithm was 99% accurate (FIG. 8B) and correctly identified 92% of the true negative images as not containing a retained foreign body object. Both the training and validation losses (mean squared error) were low at 0.00087 and 0.0018, respectively.

Specifically, FIG. 8 depicts an accuracy assessment. FIG. 8A provides a distribution of Intersection over Union (IoU) values of the test set is shown for each implanted cotton ball 290 size. Vertical boxplot lines indicate the 10th and 90th percentiles, while the boxplot itself indicates the 25th, 50th, and 75th percentiles. In FIG. 8B, the percentage of predictions with IoU values greater than 50% is shown. Here the percentage of accurately predicted bounding boxes are split by cotton ball size.

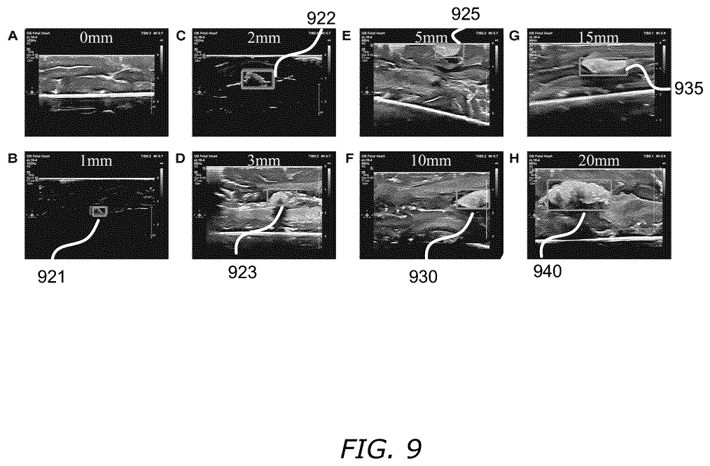

When the training and validation losses are similar to each other and low values, the algorithm performs well on all images, whether or not it has “seen” the image before [30]. Example predictions of bounding boxes on the ultrasound images 405 are shown in FIG. 9.

Specifically, FIG. 9 provides example predictions. (A) No cotton ball is present in this image, nor is one predicted to be present. Predictions of implanted cotton balls with diameter sizes of (B) 1 mm, (C) 2 mm, (D) 3 mm, (E) 5 mm, (F) 10 mm, (G) 15 mm, and (H) 20 mm in a fresh porcine brain model are shown by bounding boxes with reference values of, respectively 921 (1 mm diameter cotton ball 290), 922 (2 mm diameter cotton ball 290), 923 (3 mm diameter cotton ball 290), 925 (5 mm diameter cotton ball 290), 930 (10 mm diameter cotton ball 290), 935 (15 mm diameter cotton ball 290), and 940 (20 mm diameter cotton ball 290).

Stratified 5-fold cross validation of this model reported higher average results than the single reported model. As shown in Table 2, the mean IoU was 0.94 (from 0.93, 0.93, 0.94, 0.95, and 0.95, which were the separate models' means), while each of the sensitivity, specificity, and accuracy rounded from four significant figures to 100%.

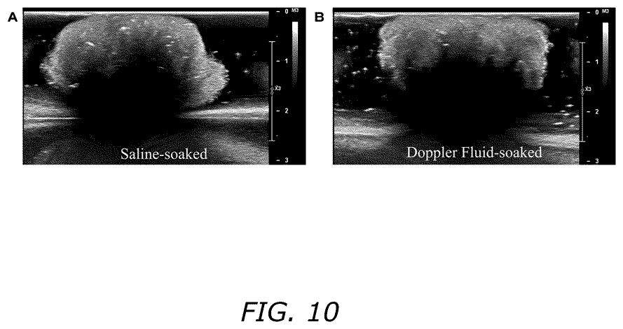

Cotton balls soaked in saline were visually similar to those soaked in blood-mimicking Doppler fluid when captured using ultrasound imaging (see FIG. 10). A visual comparison was used to avoid assuming which features were identified by the deep learning algorithm, which uses hidden layers to locate foreign objects. It was also noted that there was only a 1.5% difference in average pixel value between the lighter cotton regions on the ultrasound images as displayed in FIG. 10.

Specifically, FIG. 10 provides an acoustic comparison of cotton balls 290. In FIG. 10A, experiments were performed with saline-soaked cotton balls. In FIG. 10B, to ensure that cotton would not be visualized differently on an ultrasound machine when absorbing blood rather than saline, the saline-soaked cotton balls were compared to Doppler fluid-soaked cotton balls. Doppler fluid mimics the acoustic properties of blood. (A, B) are visually similar.

Although the speed of sound through Doppler fluid (1,570 m/s [31], CIRS, Norfolk, VA, USA) is faster than that of saline solution (approximately 1,500 m/s [32]), these fluids are comparable to the speed of sound through brain tissue (1,546 m/s [33]) but importantly are distinctly different when compared to the speed of sound through a cotton thread (3,130 m/s [34]). The high speed of sound through cotton implies that the fluid in which this material is soaked would have little influence on its visualization via ultrasound imaging. Although typically Doppler fluid is used to measure flow, the comparison between the acoustic properties of blood and Doppler fluid also indicates that these fluids are similar when stagnant as well, which would be the case during a surgery. Blood and Doppler fluid have similar speeds of sound (1,583 and 1,570 m/s, respectively), densities (1,053 and 1,050 kg/m3, respectively), attenuation coefficients (0.15 and 0.10 dB/(cm MHz), respectively), viscosities (3 and 4 mPas, respectively), particle sizes (7 and 5 mm, respectively), and backscatter coefficients (0 and 1030, respectively) [31,35,36]. They differ primarily in that blood is non-Newtonian whereas Doppler fluid is Newtonian, though this characteristic does not affect intraoperative ultrasound imaging when the blood is stagnant in the cranial cavity [36]. As a result, it is understood that the echo generated by still Doppler fluid would accurately represent an echo generated by blood.

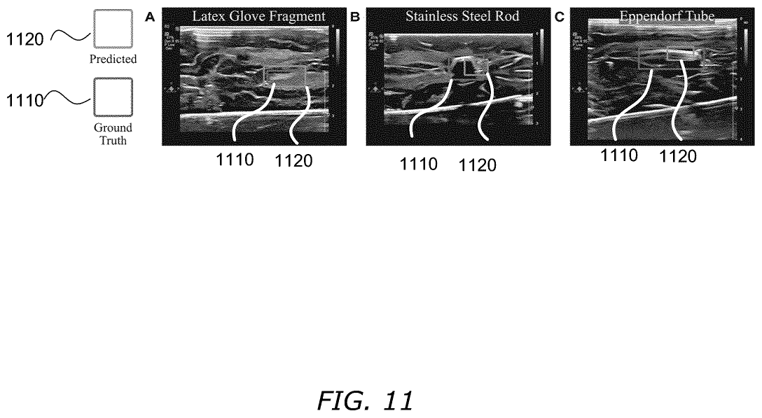

The algorithm, without any changes or additional training, was also able to detect other objects placed in or around the brain. As shown in FIG. 11, it localized a fragment of a latex glove with an IoU of 0.88, a short rod of stainless steel with an IoU of 0.68, and an Eppendorf tube with an IoU of 0.40. Although these objects were visually distinguishable from the brain tissue unlike cotton balls, this experiment proves that the ultrasound-based technology described here is beneficial in numerous use cases.

Specifically, FIG. 11 depicts the algorithmic implementation using other materials. The trained model 400, without any changes, was used to detect (FIG. 11A) latex glove fragments, (FIG. 11B) a stainless steel rod, and (FIG. 11C) an Eppendorf tube implanted into the brain. The ground truth bounding box 1110 and predicted bounding box 1120 are shown in each image.

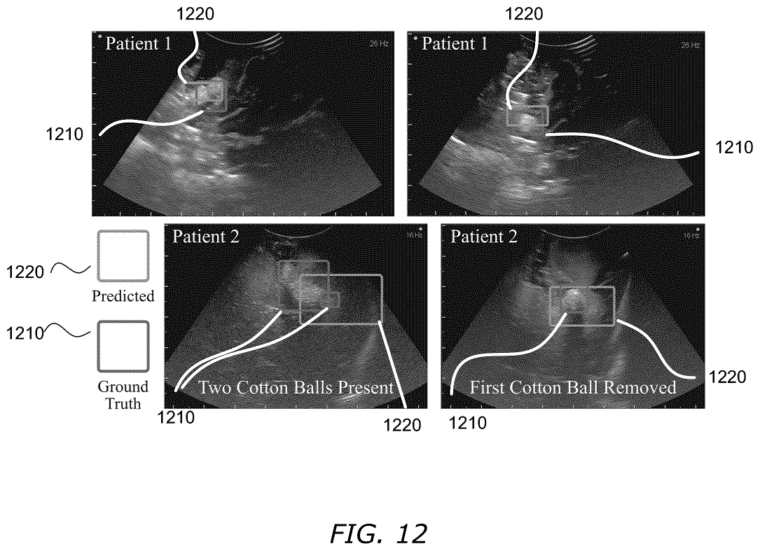

Importantly, the algorithm demonstrated the ability to prevent accidental foreign body retention and to detect cotton balls in ultrasound images captured during human neurosurgical procedures. The cotton balls placed deliberately for visualization via ultrasound during the cases (one per case) were accurately identified (see FIG. 12).

Specifically, as depicted in FIG. 12, the trained model 400, without any changes beyond initial contrast enhancement of the images, was used to detect cotton balls 290 in a human brain following neurosurgery. The top two example images were taken towards the end of an aneurysm surgery. The bottom two images were captured following a tumor resection procedure. One cotton ball was known to be placed within the cavity (left), and upon removal of this object a second cotton ball was found (right). Embodiments consistent with this disclosure prevented accidental retention of an unidentified foreign body object. Intentionally laced cotton balls had a diameter of 10 mm prior to placement within the cavity, which alters the shape. The initially unseen cotton ball in Patient 2 was 5 mm in diameter.

During the second case (Patient 2), when intending to capture a true negative image, an initially unidentified foreign body object was able to be seen in the operation site. This final ultrasound scan informed the neurosurgeons that they should explore the cavity once again. Following an extensive search, a small cotton ball approximately 5 mm in diameter was located underneath a gyral fold. This patient, undergoing a second brain surgery already this year, was protected from a third surgery that could have resulted from a retained cotton ball. This algorithm was tested post-operatively on the images captured during this surgery and accurately located both cotton balls (FIG. 12). From left to right, the IoUs of the example images for Patient 1 in FIG. 12 were 0.86 and 0.91. The IoUs of Patient 2's image with two cotton balls present were 0.72 for the larger cotton ball and 0.69 for the smaller, hidden cotton ball. The image on the right-hand side displays only this smaller, once hidden cotton ball, and the algorithm predicted its presence with an IoU of 0.83. The algorithm was unable to identify true negative images for the human studies. FIG. 12 also depicts the ground truth bounding box 1210 and the predicted bounding box 1220 in each image.

However, the Aloka UST-9120 probe used to capture these images has an operating frequency of 7 MHz, compared to the Philips eL 18-4 operating frequency of 11 MHz. Decreased frequency corresponds to lower resolution, thus indicating an approximately 50% loss in image quality of the human study compared to the ex vivo study.

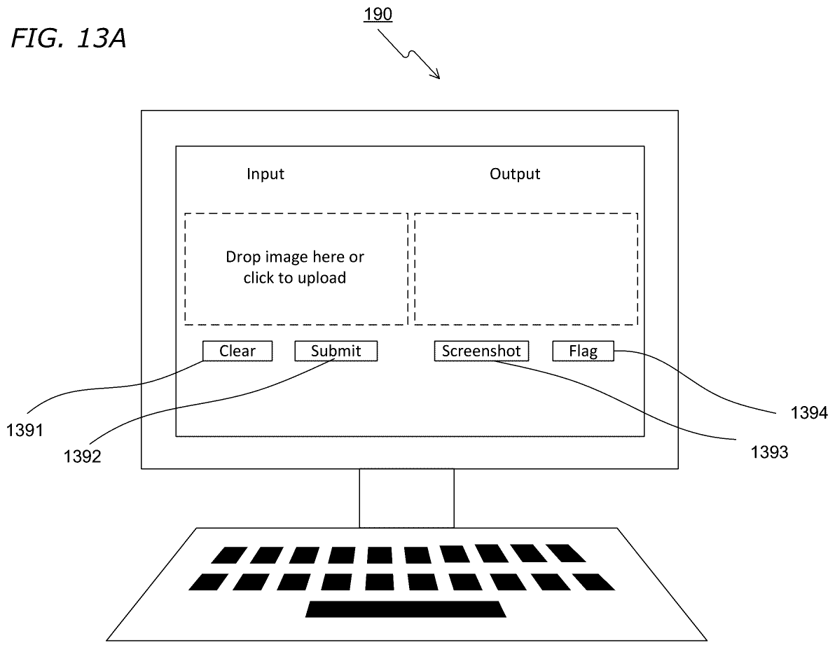

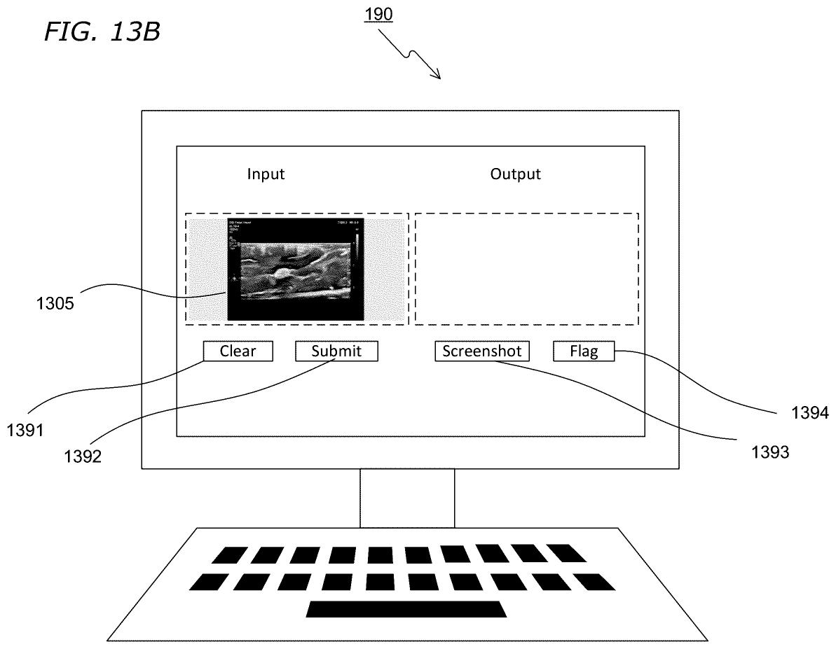

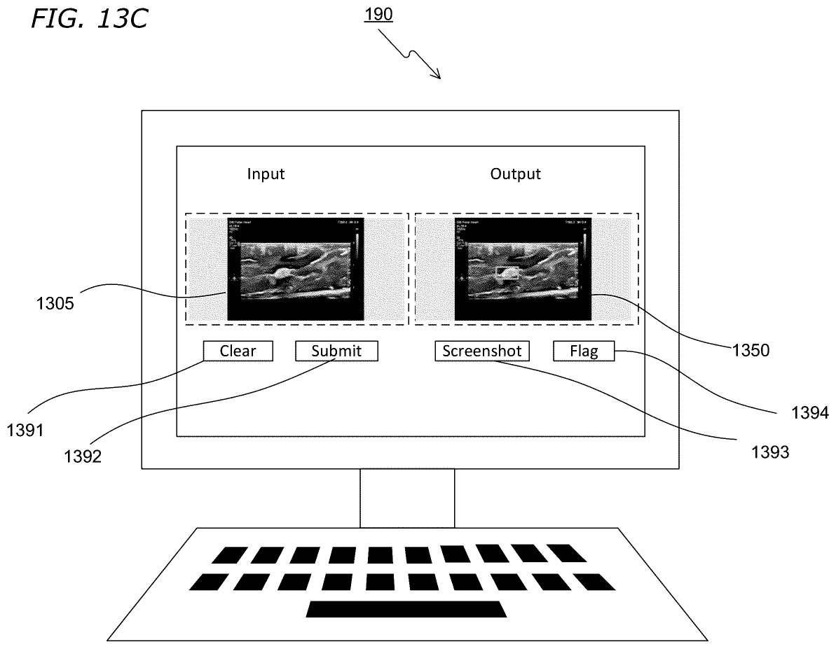

The algorithm was implemented into intuitive web and smartphone applications. A clinician may upload an image to either application, after which the application runs the trained algorithm in the back-end. In 0.38 s, the web application is able to predict, localize, and display bounding boxes on the captured ultrasound images (see FIGS. 13A-C). Specifically, in FIG. 13A, computer 190 is depicted, and in the depicted screen is a rectangular box where an image can be “dropped” or uploaded. In FIG. 13B an ultrasound image 1305 has been dropped or uploaded into the region. The buttons “Clear” (1391) or “Submit” (1392) can be selected consistent with this disclosure. “Clear” would simply reset the screen to the state shown in FIG. 13A. Clicking or activating “Submit” would submit image 1305 for processing consistent with this disclosure. That processing could be carried out either in computer 190, or the image could be transmitted to another computer or server configured to run the customized convolutional neural network 400 on the image 1305. In either case, FIG. 13C depicts the outcome with image 1350 included a predicted bounding box. The buttons “Screenshot” (1393) and “Flag” (1394) are self-explanatory.

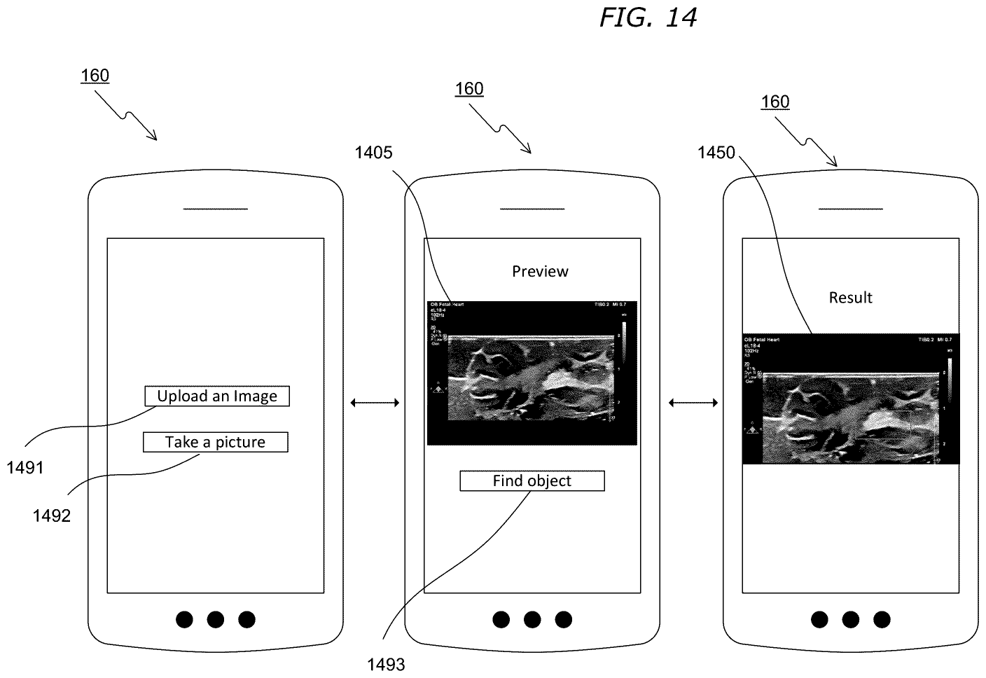

FIG. 14 depicts an implementation consistent with this disclosure on a smartphone 160. Specifically, in FIG. 14A (leftmost drawing), in the depicted screen a user has an option to “Take a picture” (1492) or to “upload an image” (1491). That is, the smartphone application offers the additional feature of being able to capture an image (i.e., “take a picture”) of the screen of an ultrasound machine and immediately check this image for cotton balls, running in approximately 1 s. In the middle drawing, an ultrasound image 1405 has been “taken” or uploaded into the phone. The button “Find object” (1493) can be selected consistent with this disclosure to subject image 1405 to processing consistent with this disclosure. That processing could be carried out either in smartphone 160, or the image could be transmitted to another computer or server configured to run the customized convolutional neural network 400 on the image 1405. In either case, the rightmost drawing depicts the outcome with image 1450 included a predicted bounding box.

### Discussion

The ultrasound-based technology presented here identifies cotton balls in the absence of injections, dyes, or radiofrequency tags and is based on clinical workflow. Cotton balls, a common item used in the operating room, serve as a model for foreign body objects that may lead to severe immunologic responses if retained post-surgery. Overcoming the visual barriers of distinguishing blood-soaked cotton from brain tissue, ultrasound imaging captured what other modalities could not: the contrasting acoustic properties of cotton in relation to brain tissue. Using thousands of acquired ex vivo porcine brain images demonstrating this contrast, a deep neural network learned the unique features of cotton in an ultrasound image and successfully output bounding boxes to localize the foreign bodies with a median IoU of 0:94+0:09 and 99% accuracy. This algorithm automated the translation of over 700,000 data points (the number of pixels in each image prior to preprocessing) to four simple numbers describing the location and size of a retained surgical item in the brain. Because gossypibomas may result from fragments of cotton [37], the work here takes caution in localizing pieces of cotton down to 1 mm in diameter. The potentially life-saving capability of embodiments consistent with this disclosure was exhibited explicitly during the second in-human data collection. The neurosurgeons had placed a cotton ball, taken an ultrasound scan, and subsequently removed it, yet there remained an unidentified foreign body object clearly visible in the image. Upon searching, they located a cotton ball that had been tucked behind a gyral fold and not initially seen by the surgeon. This object was found because they elected to perform an intraoperative ultrasound. In the future, implementing the algorithm developed here will ensure rapid and confident diagnosis of a retained foreign object.

There has only been one previous report of an algorithm for the automatic detection of foreign body objects [15]. However, the dataset acquired in Mahapatra et al. [15] was unrepresentative of a clinical setting and showed minimal variation between images, which risks overfitting. In contrast, the work described here captured all images in a manner more conducive to deep learning: sizes and locations of implanted cotton in the brain were all varied, and deformation of cotton as it absorbed saline additionally added shape variability to the images. Another benefit of this work is that all ex vivo images were acquired in a rubber-lined container to attenuate noise and avoid artifacts. Additionally, this technology is intended for clinical implementation; therefore an ultrasound machine readily available and approved for hospital use, a Philips EPIQ 7, was used. Further, this algorithm accurately localizes any size cotton ball without the added computational expense of labeling cotton size as in YOLOv4, which was used in Mahapatra et al. [15], since this label is redundant in medical images with known scales. To show that the custom neural network described here improved upon Mahapatra et al. [15], the backbone of YOLOv4 (i.e., CSPDarknet53) was trained and tested on the newly acquired image dataset. YOLOv4 is typically implemented to identify multiple different types (or classes) of objects in an image, and therefore is computationally expensive in comparison to our smaller, custom network. CSPDarknet53 is specific to localization rather than classification. Therefore, because the specific task here is to localize cotton balls rather than distinguish or classify different objects within the cranium, we did not re-implement the additional layers (known as the neck and head) of YOLOv4. CSPDarknet53 was approximately half as accurate as our custom network. Embodiments consistent with this disclosure also demonstrated the first working example of automated foreign body object detection in humans.

There are a few limitations to this work that serve as future steps in establishing this technology in the clinic. Currently, the algorithm will identify only one cotton ball per image. If there are two, for example, it will identify one of them, and upon its extraction out of the brain, identify the other. Clumped cotton balls also appear to the neural network as one singular, larger object as demonstrated in FIG. 12 Patient 2; though importantly, it recognized the presence of a foreign body. Future work should allow for multi-object detection. In addition, a few modifications can make for improved clinical translation following the first two successful implementations in humans reported here. For example, a database of ultrasound images with cotton balls used during human neurosurgery should be acquired and tested with a fine-tuned version of the algorithm presented. This database should incorporate all of the brands of clinical machines and types of probes one might find in a neurosurgical setting. The somewhat low accuracy of in vivo data compared to ex vivo results is likely due to the decreased image quality, which was cut in half in vivo due to unavailability of a more modern system or higher frequency probe, and due to the use of a curvilinear probe as opposed to linear, which the algorithm had never seen in training.

Foreign body objects could be localized using this algorithm regardless of the anatomical region, for example in abdominal, vascular, or orthopedic procedures, etc. [38-43]. Beyond cotton balls, ceramic, silicone, metal, or hydrogel implants may trigger foreign body responses that demand prompt care [44,45]. One of the first steps in treatment would be localization of the foreign body object, which could be accomplished with this technology. Using embodiments consistent with this disclosure, the ex vivo data collected demonstrated the same accuracy, sensitivity, and specificity whether or not images were filtered in pre-processing, though the methods used show promise in increasing accuracy when blurrier or poorer quality images were captured such as the in vivo data. As was demonstrated by the detection of other foreign bodies and success in humans, this algorithm is flexible as trained, and its applications could be expanded using simple fine-tuning methods. Anatomical modifications that may have occurred during surgery, which one might imagine could impact clinical translation, did not cause a noticeable issue. This algorithm searches for cotton rather than patterns in brain tissue, and neurosurgeons are unlikely to considerably change the gyral folds that may be present. Additionally, the neurosurgeon added saline to the cranial cavity, thereby removing any potential air gaps that could distort the images in vivo. Similarly, pooled blood resulting from the surgery did not and would not effect the ultrasound images because it has a similar speed of sound as saline or water, meaning that it is anechoic or hypoechoic whereas cotton is hyperechoic. Therefore, the blood would serve to further distinguish the cotton from the surrounding anatomy. Following the scanning protocol presented ensures the entire region of interest will be covered. This work could additionally benefit ultrasound uses in industry such as nondestructive testing [46,47].

### Conclusions

Ultrasound is an inexpensive, non-ionizing, and well established imaging modality across medical fields. It provides insight into the acoustic properties of different structures in the body, including foreign objects left behind during brain surgery. This work described a rapid and accurate technology that uses ultrasound imaging and is capable of localizing such foreign objects intraoperatively in humans. The importance of this work is emphasized by the fact that a cotton ball not seen by the neurosurgeon during a human procedure was located as a result of conducting ultrasound imaging in preparing this material, thereby preventing immunologic reactions in the patient, expensive follow-up surgery, and a potential malpractice lawsuit.

One of ordinary skill in the art will appreciate that other embodiments consistent with this disclosure include, but are not limited to: (1) using either a YOLO-based neural network and/or the sliding window method to detect multiple cotton balls at once; (2) applying embodiments disclosed herein to images of a patients abdomen; (3) registering the ultrasound images to the pre-op MRI to help guide the surgeon towards the location of a cotton ball, or using a navigation-enabled ultrasound probe to show on a NeuroNav system where the probe is directed when a foreign body object is found; and (4) labeling the foreign body object found (cotton ball vs stainless steel tool vs latex glove vs metal implant, etc.)

The foregoing description has been presented for purposes of illustration. The description is not exhaustive and is not limited to precise forms or embodiments disclosed. Modifications and adaptations of the embodiments will be apparent from consideration of the specification and and practice of the disclosed embodiments.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments consistent with this disclosure will be apparent from consideration of the specification and practice of the embodiments disclosed herein. It is intended that the specification and examples be considered as exemplary only, with a true scope and spirit of the disclosed embodiments being indicated by the following claims.

### APPENDIX: REFERENCES

1. National Quality Forum. List of serious reportable events (aka SRE or “never events”) (2011). Available at: https://www.qualityforum.org/topics/sres/list_of_sres.aspx
2. Bernstein L. When your surgeon accidentally leaves something inside you. WP Company (2021). Available at: https://www.washingtonpost.com/news/toyour-health/wp/2014/09/04/when-your-surgeon-accidentally-leaves-somethinginside-you/3.
3. Eisler P. What surgeons leave behind costs some patients dearly. Gannett SatelliteInformation Network (2013). Available at: https://www.usatoday.com/story/news/nation/2013/03/08/surgery-sponges-lost-supplies-patients-fatal-risk/1969603/4.
4. Sloane T. The high cost of inaction: retained surgical sponges are draining hospital finances, harming reputations (2013). Available at: https://www. beckershospitalreview.com/quality/the-high-cost-of-inaction-retained-surgicalsponges-are-draining-hospital-finances-and-harming-reputations.html
5. Dewan M C, Rattani A, Fieggen G, Arraez M A, Servadei F, Boop F A, et al. Global neurosurgery: the current capacity, deficit in the provision of essential neurosurgical care. executive summary of the global neurosurgery initiative at the program in global surgery and social change. Br J Neurosurg. (2019) 130:1055-64. doi: 10.3171/2017.11.JNS171500
6. Hempel S, Maggard-Gibbons M, Nguyen D K, Dawes A J, Miake-Lye I, Beroes J M, et al. Wrong-site surgery, retained surgical items, and surgical fires: a systematic review of surgical never events. JAMA Surg. (2015) 150:796-805. doi: 10.1001/jamasurg.2015.0301
7. Saeidiborojeni H R, Fakheri T, Iizadi B. Intracranial foreign body granulomasimulating brain tumor: a case report. J Res Med Sci. (2011) 16 (3): 358-60. PMID, 22091258; PMCID, PMC3214347.
8. Peloquin P, Vannemreddy P S, Watkins L M, Byrne R W. Intracranial cottonball gossypiboma mimicking recurrent meningioma: report of a case with literature review for intentional, unintentional foreign body granulomas. Clin Neurol Neurosurg. (2012) 7:1039-41. doi: 10.1016/j.clineuro.2012.01.046
9. Akpinar A, Ucler N, Ozdemir C O. Textiloma (gossypiboma) mimickingrecurrent intracranial abscess. BMC Res Notes. (2015) 8:1-4. doi: 10.1186/s13104-015-1315-5
10. Ribalta T, Mccutcheon I E, Neto A G, Gupta D, Kumar A J, Biddle D A, et al. Textiloma (gossypiboma) mimicking recurrent intracranial tumor. Arch Pathol Lab Med. (2004) 128:749-58. doi: 10.5858/2004-128-749-tgmrit
11. Rogers A, Jones E, Oleynikov D. Radio frequency identification (RFID) applied to surgical sponges. Surg Endosc. (2007) 21:1235-7. doi: 10.1007/s00464-007-9308-7
12. Bechtold R, Tselepidakis N, Garlow B, Glaister S, Zhu W, Liu R, et al. Minimizing cotton retention in neurosurgical procedures: which imaging modality can help? Medical Imaging 2020: Biomedical Applications in Molecular, Structural, Functional Imaging (2020) 11317:17-24. doi: 10.1117/12. 2548847
13. Kaur A, Singh Y, Neeru N, Kaur L, Singh A. A survey on deep learning approaches to medical images, a systematic look up into real-time object detection. Arch Comput Methods Eng. (2021) 29:2071-111. doi: 10.1007/s11831021-09649-9
14. Bochkovskiy A, Wang C-Y, Liao H-Y M. Yolov4: optimal speed, accuracy of object detection. arXiv preprint [arXiv: 2004.10934] (2020). Available at: https://doi.org/10.48550/arXiv.2004.10934 (Accessed Apr. 23, 2020).
15. Mahapatra S, Balamurugan M, Chung K, Kuppoor V, Curry E, Aghabaglou F, et al. Automatic detection of cotton balls during brain surgery: where deep learning meets ultrasound imaging to tackle foreign objects. Medical Imaging 2021: Ultrasonic Imaging, Tomography (2021) 11602:295-302. doi: 10.1117/12.2580887
16. Weickenmeier J, Kurt M, Ozkaya E, de Rooij R, Ovaert T C, Ehman R, et al. Brain stiffens post mortem. J Mech Behav Biomed Mater. (2018) 84:88-98. doi: 10.1016/j.jmbbm.2018.04.009
17. Toennies K D, Guide to medical image analysis. London: Springer (2017).
18. Otsu N. A threshold selection method from gray-level histograms. IEEE Trans Syst Man Cybern. (1979) 9:62-6. doi: 10.1109/TSMC.1979.4310076
19. Rosebrock A. Multi-scale template matching using python and OpenCV (2015). Available at: https://pyimagesearch.com/2015/01/26/multi-scaletemplate-matching-using-python-opencv/20.
20. Wang C, Liao H M, Yeh I, Wu Y, Chen P, Hsieh J. CSPNet: a new backbone that can enhance learning capability of CNN. CORR (2019). abs/1911.11929
21. Simonyan K, Zisserman A. Very deep convolutional networks for large-scale image recognition. arXiv preprint [arXiv: 1409.1556] (2015). Available at: https://doi.org/10.48550/arXiv.1409.1556 (Accessed Sep. 4, 2014).
22. Chollet F, Deep learning with Python. Shelter City, NY: Simon and Schuster (2021).
23. Chollet F, et al. Keras. GitHub (2015). Available at: https://github.com/fchollet/keras
24. Kingma D, Ba J. Adam: a method for stochastic optimization. International Conference on Learning Representations. arXiv preprint [arXiv: 1409.1556] (2014). Available at: https://doi.org/10.48550/arXiv.1412.6980 (Accessed Dec. 22, 2014).
25. Everingham M, Van Gool L, Williams C K I, Winn J, Zisserman A. The pascalvisual object classes (VOC) challenge. Int J Comput Vis. (2010) 88:303-38. doi: 10. 1007/s11263-009-0275-4
26. Abid A, Abdalla A, Abid A, Khan D, Alfozan A, Zou J Y. Gradio: Hassle-freesharing and testing of ML models in the wild. CoRR (2019). abs/1906.02569
27. Google. Dart. GitHub (2012). Available at: https://github.com/dart-lang
28. Abadi M, Agarwal A, Barham P, Brevdo E, Chen Z, Citro C, et al. TensorFlow: large-scale machine learning on heterogeneous systems (2015). Software available from tensorflow.org.
29. Rosebrock A. Object detection: bounding box regression with Keras, TensorFlow, and deep learning (2021). Available at: https://pyimagesearch.com/2020 Oct. 5/object-detection-bounding-box-regression-with-keras-tensorflow-anddeep-learning/30.
30. Dietterich T. Overfitting and undercomputing in machine learning. ACM Comput Surv (CSUR). (1995) 27:326-7. doi: 10.1145/212094.212114
31. CIRS. Doppler fluid (2021). Available at: https://www.cirsinc.com/products/ultrasound/doppler-fluid/
32. Encyclopaedia Britannica. Acoustic properties. Encyclopaedia Britannica (2019). Available at: https://www.britannica.com/science/seawater/Acousticproperties
33. Hasgall P A, Di Gennaro F, Baumgartner C, Neufeld E, Lloyd B, Gosselin M,Payne D, et al. IT′IS database for thermal and electromagnetic parameters of biological tissues. Tissue Properties: Speed and Sound (2022). doi: 10.13099/VIP21000 Apr. 1. Available at: itis.swiss/database
34. Saito S, Shibata Y, Ichiki A, Miyazaki A. Measurement of sound speed inthread. Jpn J Appl Phys. (2006) 45:4521-5. doi: 10.1143/jjap.45.4521
35. Supertech. Doppler flow pump-CIRS 769: perform sensitivity and velocity QA on doppler ultrasound (2022). Available at: https://www.supertechx-ray.com/Ultrasound/DopplerPhantoms/cirs-doppler-flow-pump-769.php
36. Samavat H, Evans J. An ideal blood mimicking fluid for doppler ultrasound phantoms. J Med Phys. (2006) 31:275. doi: 10.4103/0971-6203.29198
37. Kim A, Lee E, Bagley L, Loevner L. Retained surgical sponges after craniotomies: imaging appearances and complications. Am J Neuroradiol. (2009) 30:1270-2. doi: 10.3174/ajnr.A1469
38. Stawicki S P, Evans D, Cipolla J, Seamon M, Lukaszczyk J, Prosciak M, et al., Retained surgical foreign bodies: a comprehensive review of risks and preventive strategies. Scand J Surg. (2009) 98:8-17. doi: 10.1177/145749690909800103
39. Kastiunig T, Sortino R, Vines L C, Benigno L. Intra-abdominal foreign bodyas unexpected discovery mimicking suspicious malignancy. J Surg Case Rep. (2021) 2021 (6): rjab248. doi: 10.1093/jscr/rjab248
40. Zarenezhad M, Gholamzadeh S, Hedjazi A, Soltani K, Gharehdaghi J, Ghadipasha M, et al. Three years evaluation of retained foreign bodies after surgery in Iran. Ann Med Surg. (2017) 15:22-5. doi: 10.1016/j.amsu.2017. 01.019
41. Pyeon T, Bae H-B, Choi J I, Kim T, Kim J. Incidental detection of a retained left atrial catheter via intraoperative transesophageal echocardiography in a patient undergoing tricuspid valve replacement: a case report. Medicine (2020) 99 (19): e20058. doi: 10.1097/MD.0000000000020058
42. Whang G, Lekht I, Krane R, Peters G, Palmer S L. Unintentionally retained vascular devices: improving recognition and removal. Diagn Interv Radiol. (2017) 23:238. doi: 10.5152/dir.2017.16369
43. Franco C, Moskovitz A, Weinstein I, Kwartin S, Wolf Y. Long term rigid retained foreign object after breast augmentation: a case report and literature review. Front Surg. (2021) 8:725273. doi: 10.3389/fsurg.2021.725273
44. Veiseh O, Doloff J C, Ma M, Vegas A J, Tam H H, Bader A R, et al. Size-andshape-dependent foreign body immune response to materials implanted in rodents and non-human primates. Nat Mater. (2015) 14:643-51. doi: 10.1038/nmat4290
45. Doloff J C, Veiseh O, de Mezerville R, Sforza M, Perry T A, Haupt J, et al. The surface topography of silicone breast implants mediates the foreign body response in mice, rabbits and humans. Nat Biomed Eng. (2021) 5:1115-30. doi: 10.1038/s41551-021-00739-4
46. Bombarda D, Vitetta G M, Ferrante G. Rail diagnostics based on ultrasonic guided waves: an overview. Appl Sci. (2021) 11:1071. doi: 10.3390/app 11031071
47. Heyman J S. Applications of ultrasonics in aerospace. Ultrason Int. (1983) 83:1. doi: 10.1016/b978-O-408-22163-4.50004-0

1. A method of forming a trained model in which one or more processing devices perform operations comprising:
receiving a plurality of training images, each training image in the plurality of training images associated with a respective training boundary data set of a plurality of training boundary data sets, each training image in the plurality of training images further represented as a respective training image data set of a plurality of training image data sets;
wherein each of the plurality of training images is a respective ultrasound image of a plurality of ultrasound images, the respective ultrasound image further associated with a respective one of a plurality of regions;
processing the plurality of training image data sets using a convolutional neural network to generate a plurality of output boundary data sets and to select a plurality of training weights;
wherein the convolutional neural network comprises (i) a plurality of pre-trained layers with a plurality of pre-trained weights, and (ii) a plurality of training and appended layers with the plurality of training weights; and
wherein, when using the convolutional neural network to generate the plurality of output boundary data sets, the plurality of pre-trained weights are fixed and the plurality of training weights are selected to minimize a loss function between the plurality of output boundary data sets and the plurality of respective training boundary data sets; and
fixing the plurality of training weights to form the trained model when the loss function is minimized;
wherein fixing the plurality of training weights further selects a plurality of fixed training weights.

2. The method of claim 1, wherein the loss function is a mean squared error function using a plurality of error values, each error value of the plurality of error values being equal to a respective numerical difference between at least one of the plurality of output boundary data sets and a respective one of the plurality of training boundary data sets.

3. The method of claim 1, wherein at least one of the plurality of ultrasound images associated with a respective one of the plurality of regions is further associated with a respective foreign body object in the respective one of the plurality of regions.

4. The method of claim 3, wherein the at least one of the plurality of ultrasound images associated with the respective one of the plurality of regions is further associated with a respective one of the plurality of training boundary data sets, such that the respective one of the plurality of training boundary data sets is a set of number values associated with a bounding box enclosing the respective foreign body object.

5. The method of claim 4, wherein the bounding box enclosing the respective foreign body object is a ground truth bounding box.

6. The method of claim 1, wherein at least one of the plurality of ultrasound images is associated with a respective one of the plurality of regions such that the respective one of the plurality of regions contains no foreign body object.

7. The method of claim 6, wherein the at least one of the plurality of ultrasound images associated with the respective one of the plurality of regions containing no foreign body object is further associated with a respective one of the plurality of training boundary data sets, such that the respective one of the plurality of training boundary data sets is a set of number values associated with a null bounding box.

8. The method of claim 7, wherein the set of number values associated with the null bounding box comprises:
an x-coordinate value equal to zero of the null bounding box;
a y-coordinate value equal to zero of the null bounding box;
a width value equal to zero of the null bounding box; and
a height value equal to zero of the null bounding box.

9. The method of claim 4, wherein the set of number values associated with the bounding box enclosing the respective foreign body object comprises:
an x-coordinate value of an upper left corner of the bounding box;
a y-coordinate value of the upper left corner of the bounding box;
a width value of the bounding box; and
a height value of the bounding box.

10. A method of generating a boundary data set from an input image in which one or more processing devices perform operations comprising:
forming the trained model of claim 9;
receiving the input image represented as an input image data set; and
processing the input image data set using the convolutional neural network to generate the boundary data set;
wherein the convolutional neural network comprises (i) the plurality of pre-trained layers with the plurality of pre-trained weights, and (ii) the plurality of training and appended layers with the plurality of fixed training weights.

11. The method of claim 10, wherein the boundary data set is a set of output number values associated with an output bounding box enclosing a localized region in the input image, the set of output number values associated with the output bounding box enclosing the localized region comprising:
an output x-coordinate value of an upper left corner of the output bounding box;
an output y-coordinate value of the upper left corner of the output bounding box;
an output width value of the output bounding box; and
an output height value of the output bounding box.

12. The method of claim 11, wherein the convolutional neural network comprises a VGG16 model.

13. The method of claim 12, wherein the input image is an ultrasound image of a region associated with a potential foreign body object in the region.

14. The method of claim 13, wherein each respective foreign body object comprises at least one of: a cotton ball, a stainless steel rod, a latex glove fragment, an Eppendorf tube, a suturing needle, and a surgical tool.

15. The method of claim 11, wherein the plurality of appended layers comprise at least one of: a dropout layer and a dense layer.

16. The method of claim 11, wherein the plurality of trained layers comprise a portion of a VGG16 model.

17. The method of claim 11, further comprising:
generating a representation for display on a display device, the representation for display including an overlay of a representation of the output bounding box on a representation of the input image.

18. A system for forming a trained model, the system comprising:
a non-transitory computer readable storage medium associated with a computing device, the non-transitory computer readable storage medium storing program instructions executable by the computing device to cause the computing device to perform operations comprising:
receiving a plurality of training images, each training image in the plurality of training images associated with a respective training boundary data set of a plurality of training boundary data sets, each training image in the plurality of training images further represented as a respective training image data set of a plurality of training image data sets;
wherein each of the plurality of training images is a respective ultrasound image of a plurality of ultrasound images, the respective ultrasound image further associated with a respective one of a plurality of regions;
processing the plurality of training image data sets using a convolutional neural network to generate a plurality of output boundary data sets and to select a plurality of training weights;
wherein the convolutional neural network comprises (i) a plurality of pre-trained layers with a plurality of pre-trained weights, and (ii) a plurality of training and appended layers with the plurality of training weights; and
wherein, when using the convolutional neural network to generate the plurality of output boundary data sets, the plurality of pre-trained weights are fixed and the plurality of training weights are selected to minimize a loss function between the plurality of output boundary data sets and the plurality of respective training boundary data sets; and
fixing the plurality of training weights to form the trained model when the loss function is minimized;
wherein fixing the plurality of training weights further selects a plurality of fixed training weights.

19-26. (canceled)

27. A system comprising:
at least one processor; and
at least one non-transitory computer readable media associated with the at least one processor storing program instructions that when executed by the at least one processor cause the at least one processor to perform operations for generating a boundary data set from an input image, the operations comprising:
receiving the input image represented as an input image data set; and
processing the input image data set using a convolutional neural network to generate the boundary data set;
wherein the convolutional neural network comprises (i) a plurality of pre-trained layers with a plurality of pre-trained weights, and (ii) a plurality of training and appended layers with a plurality of fixed training weights;
wherein the plurality of fixed training weights are selected according to training operations performed by one or more processors associated with one or more non-transitory computer readable media, the one or more non-transitory computer readable media storing training program instructions that when executed by the one or more processors cause the one or more processors to perform the training operations comprising:
receiving a plurality of training images, each training image in the plurality of training images associated with a respective training boundary data set of a plurality of training boundary data sets, each training image in the plurality of training images further represented as a respective training image data set of a plurality of training image data sets;
wherein each of the plurality of training images is a respective ultrasound image of a plurality of ultrasound images, the respective ultrasound image further associated with a respective one of a plurality of regions;
processing the plurality of training image data sets using a training convolutional neural network to generate a plurality of output boundary data sets and to select a plurality of training weights;
wherein the training convolutional neural network comprises (i) the plurality of pre-trained layers with the plurality of pre-trained weights, and (ii) the plurality of training and appended layers with the plurality of training weights; and
wherein, when using the training convolutional neural network to generate the plurality of output boundary data sets, the plurality of pre-trained weights are fixed and the plurality of training weights are selected to minimize a loss function between the plurality of output boundary data sets and the plurality of respective training boundary data sets; and
fixing the plurality of training weights to form the trained model when the loss function is minimized;
wherein fixing the plurality of training weights further selects the plurality of fixed training weights.

28-42. (canceled)

43. The system of claim 42, wherein a smartphone comprises the at least one processor, the at least one non-transitory computer readable media, and the display device, wherein the smartphone is further configured to capture the input image, or wherein the system further comprises:
a networked computer device that comprises the at least one processor and the at least one non-transitory computer readable media; and
a remote computing device that comprises the display device and that is configured to transmit the input image to the networked computing device, wherein the remote computing device is further configured to capture the input image, wherein the remote computing device is a smartphone.

44-47. (canceled)
